(19) United States
(12) Patent Application Publication
(10) Pub. No.: US 2009/0143585 A1
(43) Pub. Date: Jun. 4, 2009

(54) BIFUNCTIONAL CATALYSTS FOR EXTENSIVE ISOMERIZATION OF UNSATURATED HYDROCARBONS

(76) Inventor: Douglas Grotjahn, San Diego, CA (US)

Correspondence Address: CATALYST LAW GROUP, APC, 9710 SCRANTON ROAD, SUITE S-170, SAN DIEGO, CA, 92121, US

(21) Appl. No.: 12/067,790

(22) PCT Filed: Sep. 21, 2006
(86) PCT No.: PCT/US06/36931

§ 371 Date: Oct. 7, 2008

Related U.S. Application Data
(60) Provisional application No. 60/719,807, filed on Sep. 21, 2005.

(51) Int. Cl. C07C 29/56; C07F 15/00

(52) U.S. Cl. 546/4; 548/103; 568/909.5

(57) ABSTRACT

The current invention provides novel bifunctional catalysts. The bifunctional catalysts are prepared from phosphine ligands and a cyclopentadienyl metal complex and are useful for forming isomers of hydrocarbon species. The hydrocarbon can be an alkenol having the alkene and alcohol groups far apart and the catalyst will move the double bond across numerous carbon atoms. The hydrocarbon can also be an achiral alkenol and the catalyst will form a chiral alcohol therefrom. Moreover, deuterated water may be added to the isomerization reaction mixture for forming deuterated hydrocarbon species.

### FIELD OF THE INVENTION

This invention relates generally to the field of bifunctional catalysts prepared using phosphine ligands comprising pendant acids or bases in the vicinity of a metal center.

### BACKGROUND

In the prior art there are a number of compositions and methods for harnessing the ability of a transition metal to migrate a double bond across a hydrocarbon chain. It is typically the group 8, 9 and 10 transitions metals that are employed for this transformation. A variety of ruthenium derivatives have been used for isomerization reactions. For the transposition of methallyl alcohol to isobutyraldehyde it is common to use 0.6 mol % of the catalyst RuCl.sub.3 and trifluoroethanol at 70 .deg.C. Similarly, using a 1:1 ratio of RuCl.sub.3 and NaOH, a quantative isomerization reaction can be performed on allylic alcohols and glycols. Furthermore, using chiral nonracemic alcohols transposition occurs with significant chirality transfer.

Other catalysts using ruthenium include Ru(acac).sub.3, which isomerizes a wide range of 1-substituted propenes; Ru(H.sub.2O).sub.6(tos).sub.2, which rearranges simple allylic ethers and alcohols; Ru.sub.3O(OCOCH.sub.3).sub.7, which is useful for the transposition of simple secondary alcohols; and CpRu(PPh.sub.3).sub.2Cl, which is useful for isomerizing cinnamyl alcohols and allylic secondary alcohols. The migration of remote double bonds using catalysts of the prior art is at a much lower rate compared to the allyl alcohols. Thus there is a need in the art for more efficient catalysts

### SUMMARY OF THE INVENTION

One embodiment of the present invention relates to novel bifunctional catalysts. The bifunctional catalysts are prepared from phosphine ligands and a cyclopentadienyl metal complex.

In one particular aspect of the present invention, the catalysts are useful for forming isomers of hydrocarbon species.

The hydrocarbon can be an alkenol having the alkene and alcohol groups far apart and the catalyst will move the double bond across numerous carbon atoms.

The hydrocarbon can be an achiral alkenol and the catalyst forms a chiral alcohol therefrom.

Moreover, deuterated water may be added to the isomerization reaction mixture for forming deuterated hydrocarbon species.

### DETAILED DESCRIPTION OF THE INVENTION

The current invention describes a bifunctional catalyst that is created using phosphines or other ligands containing pendant bases or acids in the vicinity of the metal center. Preferably the ligand is heterocyclic. These catalysts are useful for isomerization of unsaturated hydrocarbons. One particular advantage of the current invention catalysts is that they are particularly active for isomerizing alkenols in which the alkene and the alcohol groups are far apart. Because of the catalysts' high activity, the mole ratio of catalyst to substrate is substantially reduced as compared to the typical 1:1 ratio using the prior art catalysts. Moreover, the invention catalysts can move the double bond of an allyl alcohol a much greater distance than can the prior art compounds.

The catalysts include a transition metal atom, M, (e.g. ruthenium) surrounded by ligands. Ligands for good catalytic performance include not only atom(s) to bind to the metal, but also atom(s) which can act as bases or acids. Without being held to any theory of these catalysts' actions, it is believed that the combined action of the transition metal and the bases or acids in the same molecule are what create the uniquely powerful and efficient catalysts for moving double bonds in organic molecules.

Catalysts can generally be prepared as shown in Scheme I by using a cyclopentadienyl-metal complex (CpM) and an imidazol-2-yl phosphine ligand to give the catalyst structure of Formula I.

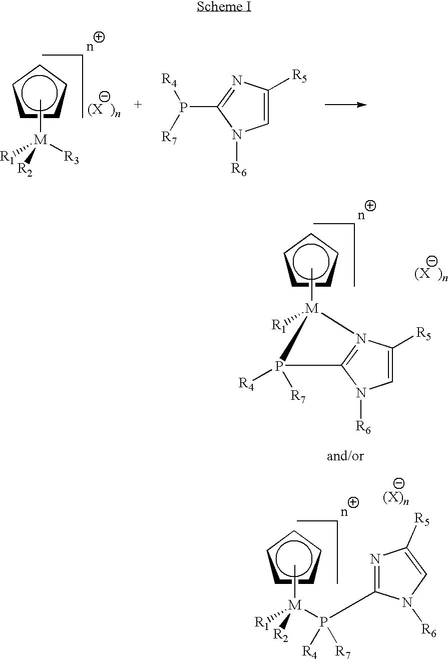

Wherein: R1 can be CH.sub.3CN or derivatives thereof, halide, hydride, carboxylate, sulfonate, or any substituted derivatives thereof, or any neutral or anionic ligand.

R2 can be CH.sub.3CN or derivatives thereof, halide, hydride, carboxylate, sulfonate, or any substituted derivatives thereof, or any neutral or anionic ligand.

R3 can be CH.sub.3CN or derivatives thereof, halide, hydride, carboxylate, sulfonate, or any substituted derivatives thereof, or any neutral or anionic ligand.

R4 can be CH(CH.sub.3).sub.2, C(CH.sub.3.).sub.2, or any alkyl or aryl group, including heteroaryl.

R5 can be C(CH.sub.3).sub.3, H, CH(CH.sub.3).sub.2, or any alkyl or aryl group, including heteroaryl.

R6 can be CH.sub.3, H, or any alkyl or aryl group.

R7 can be CH(CH.sub.3).sub.2, C(CH.sub.3.).sub.2, or any alkyl or aryl group, including heteroaryl.

M can be a transition metal, a 1+, 2+, or 3+ oxidation state transition metal, a group 6, 7, 8, or 9 transition metal, chromium, molybdenum, tungsten, manganese, rhenium, iron, ruthenium, osmium, cobalt, rhodium, iridium, nickel, palladium, platinum, copper, silver, or gold.

N can be 0, 1, 2, 3, 4, 5, 6, 7 or 8.

(X).sub.n can be PF.sub.6.

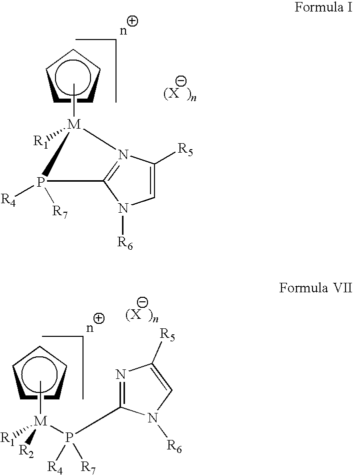

By using an alternative ligand, the catalyst can be prepared as shown in Scheme II to get the structure of Formula II.

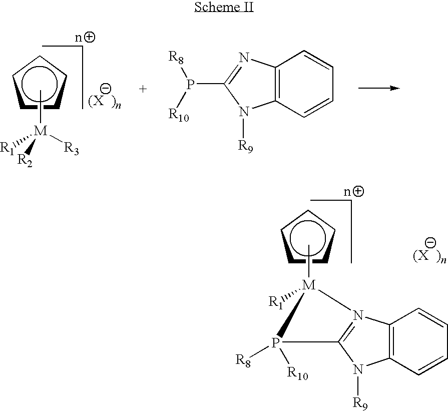

Wherein R8 can be CH(CH.sub.3).sub.2, C(CH.sub.3.).sub.2, or any alkyl or aryl group, including heteroaryl.

R9 can be CH.sub.3, H, or any alkyl or aryl group.

R10 can be CH(CH.sub.3).sub.2, C(CH.sub.3.).sub.2, or any alkyl or aryl group, including heteroaryl.

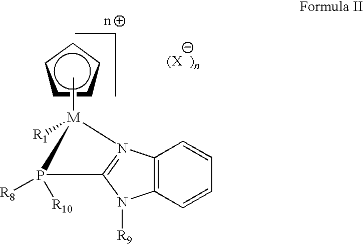

Scheme III shows the general synthesis of a catalyst by using a CpM and a pyrid-2-yl phosphine ligand to give the catalyst structure of Formula III.

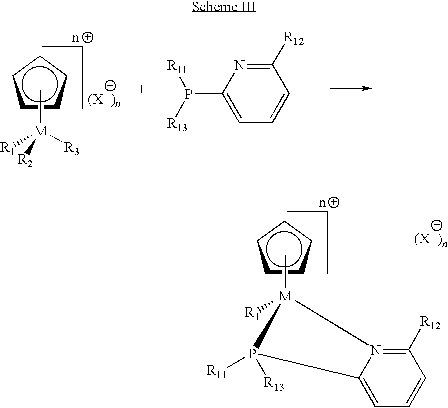

Wherein R11 can be CH(CH.sub.3).sub.2, C(CH.sub.3.).sub.2, or any alkyl or aryl group, including heteroaryl.

R12 can be C(CH.sub.3).sub.3, H, CH(CH.sub.3).sub.2, or any alkyl or aryl group, including heteroaryl.

R13 can be CH(CH.sub.3).sub.2, C(CH.sub.3.).sub.2, or any alkyl or aryl group, including heteroaryl.

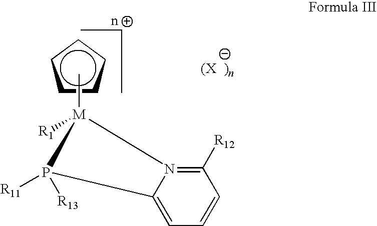

### EXAMPLE 1

In the preferred embodiment, the CpM species comprises a transition metal that is preferably Ru(2+). The bifunctional catalysts, therefore, are prepared by reacting a precursor containing the cyclopentadienyl ligand and a ruthenium(2+) ion (CpRu+) with either an imidazol-2-yl or pyrid-2-yl phosphine ligand. In Scheme IV there is provided the synthesis of the preferred embodiment for the catalyst of Formula IV reacting CpRu and an imidazol-2-yl phosphine ligand.

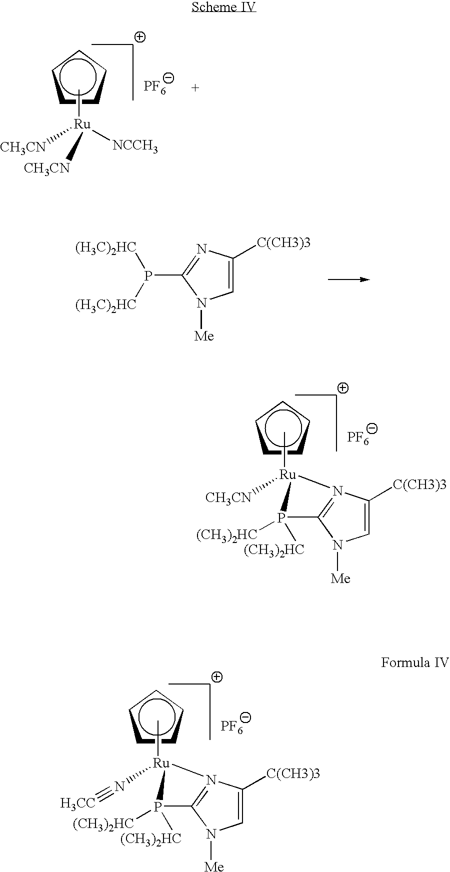

Preparation of the Formula IV catalyst [CpRu(η²-P,N-L)(CH₃CN)]PF₆.

[CpRu(CH₃CN)₃]PF₆ (296.9 mg, 0.68 mmol) was added to a scintillation vial containing a stir bar in the glove box. Dry, degassed CH₂Cl₂ (10 mL) was then added followed by the addition of the phosphine L (175.3 mg, 0.68 mmol). The mixture was allowed to stir overnight. The solvent was removed by vacuum, and to the residue was added pentane. Evaporation of solvents under vacuum led to brownish crystals. The solid was dissolved in CH₂Cl₂, followed by removal of the solvent under vacuum. This was repeated six times, until the amount of unchelated complex [CpRu(η¹-P-L)(CH₃CN)](CH₃CN)₂]PF₆ was undetectable by NMR. This process yielded [CpRu(η²-P,N-L)(CH₃CN)]PF₆ (285 mg, 91% yield). ¹H NMR (CDCl₃, 500 MHz) d 1.01 (dd, 3H, J=7.5, 16.5 Hz), 1.208 (dd, 3H, J=10.5, 18 Hz), 1.26 (dd, 3H, obscured by s at 1.30), 1.30 (s, 9H), 1.45 (dd, 3H, J=6.5, 17 Hz), 2.30 (s, 3H), 2.57-2.63 (m, 1H), 2.83-2.88 (m, 1H), 3.66 (s, 3H), 4.64 (d, 5H, J=0.5 Hz), 6.66 (s, 1H). ³¹P NMR (CD₃COCD₃, 500 MHz) d 39.43 (s).

### EXAMPLE 2

Scheme V provides the synthesis of a further example of the invention bifunctional catalyst. The catalyst was synthesized under conditions similar those described above. The catalyst is illustrated in Formula V.

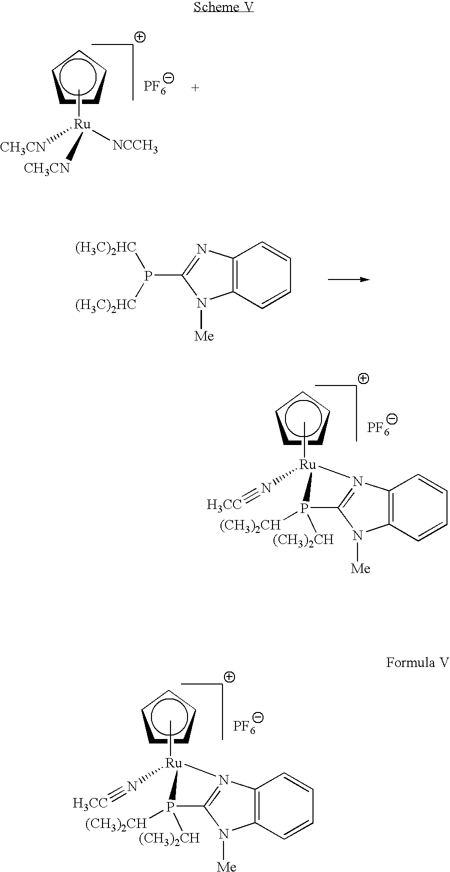

### EXAMPLE 3

Similarly, the specific catalyst of Formula VI can be formed

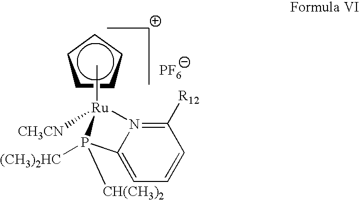

These bifunctional catalysts, derived from reacting ligands and transition metals, are useful for forming isomers of unsaturated hydrocarbons, for forming chiral aldehydes from achiral alkenols, and for forming deuterated alkenes.

### EXAMPLE 4

### Isomerization of pent-4-en-1-ol to Pentanal

In a first example showing use of the current invention catalyst, pent-4-en-1-ol is isomerized to pentanal using the catalyst of Formula IV.

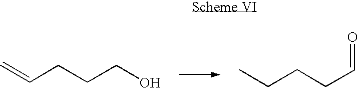

To a J. Young resealable NMR tube in the glovebox was added pent-4-en-1-ol (51.6 μL, 43 mg, 0.5 mmol) and an internal standard [(Me₃Si)₄C], and acetone-d₆ to bring the total volume to 1 mL. The proton NMR spectrum was acquired. In the glovebox, the catalyst (4.6 mg, 0.01 mmol) was added. Outside the glovebox, the NMR tube was then placed in an oil bath at 70° C. Observation of the mixture by NMR spectroscopy after 1, 2, and 5 h revealed that pentanal had been formed in over 95% yield after 5 h. ¹H NMR of the product in the mixture (CD₃COCD₃, 500 MHz) d 0.90 (t, 3H, J=7 Hz), 1.33-1.36 (m, 2H), 1.54-1.60 (m, 2H), 2.04-2.06 (m, 2H), 2.42 (dt, J=1.8, 7 Hz), 9.72 (t, 1H, J=1.8 Hz).

### EXAMPLES 5 AND 6

In a further example the catalyst of Formula V is shown isomerizing 1-pentene to a mixture of isomers within 1 hour at room temperature using only 2 mol % of catalyst (Scheme VII). It is additionally shown isomerizing 4-penten-1-ol to the aldehyde pentanal (Scheme VIII). In the pentenol case, isomerization proceeds through several stages. E- and Z-1 penten-1-ol is the most stable of the alkene isomers and then a final equilibration between the keto and enol leading to a pure aldehyde (greater than 95% yield). In these example reactions the acetone used in Scheme VI is substituted with THF (Scheme VII) and with methylene chloride (Scheme VIII). In a variation of this example reaction, it has been determined that using 5 mol % of the catalyst at room temperature allows isomerization to complete in 1 to 2 days.

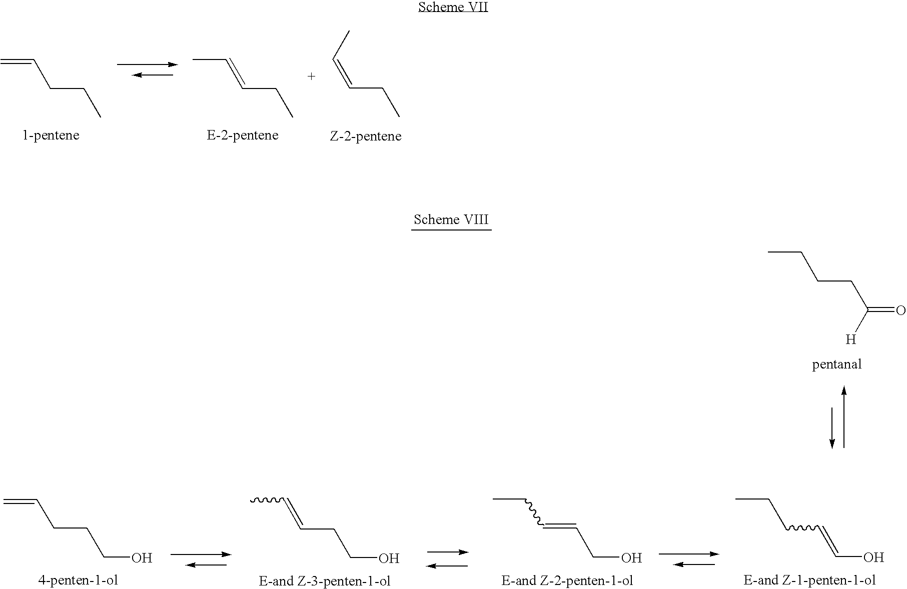

### EXAMPLE 7

In a further example using the invention bifunctional catalyst, octadec-9-en-1,18-diol can be isomerized to the unsymmetrical compound 18-hydroxyoctadecanal, a process which must involve moving the double bond past 8 carbon atoms. If one were to try performing this isomerization process using the prior art method of hydrogenating and then selectively oxidizing one alcohol only, it would be difficult or impossible to do so in over 50% yield. However, using the catalysts of the current invention, yield is over 90% without wasting any reactant. Thus, these catalysts are useful for moving a double bond across numerous carbon atoms.

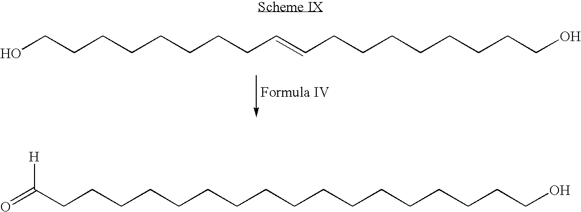

### EXAMPLE 8

Using an ether of 4-penten-1-ol (R14=tBuPh₂Si), with the catalysts of the current invention, the reaction is done within hours using 2 mol % catalyst at 70° C. and a nearly pure E isomer is formed. Formula IV catalyst is used as described above.

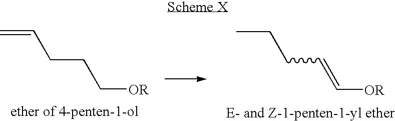

### EXAMPLE 9

In a further example showing the versatility of the current bifunctional catalysts, alkenes can be deuterated. In this example, 1-pentene is isomerized using 5 mol % catalyst (Formula IV) in the presence of 10 equiv. D₂O at room temperature. 1H NMR spectra of the mixture over time showed the complete isomerization of pentene within 1 hour followed by a slower (36 hour) incorporation of deuterium in to all positions of the alkene.

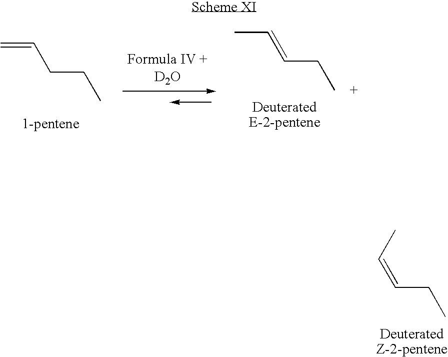

We claim:

1. A catalyst of Formula I: 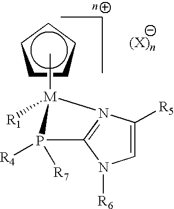
wherein R1 is selected from the group consisting of CH.sub.3CN or derivatives thereof, halide, hydride, carboxylate, sulfonate, or any substituted derivatives thereof, or any neutral or anionic ligand; R4 is selected from the group consisting of CH(CH.sub.3).sub.2, C(CH.sub.3.).sub.2, or any alkyl or aryl group, including heteroaryl; R5 is selected from the group consisting of C(CH.sub.3).sub.3, H, CH(CH.sub.3).sub.2, or any alkyl or aryl group, including heteroaryl; R6 is selected from the group consisting of CH.sub.3, H, or any alkyl or aryl group; R7 is selected from the group consisting of CH(CH.sub.3).sub.2, C(CH.sub.3.).sub.2, or any alkyl or aryl group, including heteroaryl; and M is selected from the group consisting of a transition metal, a 1+, 2+, or 3+ oxidation state transition metal, a group 6, 7, 8, or 9 transition metal, chromium, molybdenum, tungsten, manganese, rhenium, iron, ruthenium, osmium, cobalt, rhodium, iridium, nickel, palladium, platinum, copper, silver, or gold.

2. The catalyst of claim 1 wherein R1 is CH.sub.3CN; R4 is CH(CH.sub.3).sub.2; R5 is CH(CH.sub.3).sub.3; R6 is CH.sub.3; R7 is CH(CH.sub.3).sub.2; and M is Ruthenium, giving formula IV

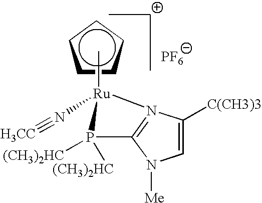

3. A method of synthesizing the catalyst of claim 1 using the steps of:
(a) utilizing a precursor containing a cyclopentadienyl ligand and a metal ion;
(b) reacting the precursor with an imidazol-2-yl phosphine ligand;
wherein the metal ion is selected from the group consisting of a transition metal, a 1+, 2+, or 3+ oxidation state transition metal, a group 6, 7, 8, or 9 transition metal, chromium, molybdenum, tungsten, manganese, rhenium, iron, ruthenium, osmium, cobalt, rhodium, iridium, nickel, palladium, platinum, copper, silver and gold.

4. A catalyst of Formula II: 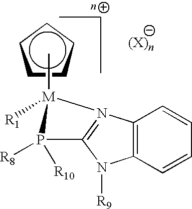
wherein R1 is selected from the group consisting of CH.sub.3CN or derivatives thereof, halide, hydride, carboxylate, sulfonate, or any substituted derivatives thereof, or any neutral or anionic ligand; R8 is selected from the group consisting of CH(CH.sub.3).sub.2, C(CH.sub.3.).sub.2, or any alkyl or aryl group, including heteroaryl; R9 is selected from the group consisting of CH.sub.3, H, or any alkyl or aryl group; R10 is selected from the group consisting of CH(CH.sub.3).sub.2, C(CH.sub.3.).sub.2, or any alkyl or aryl group, including heteroaryl; and M is selected from the group consisting of a transition metal, a 1+, 2+, or 3+ oxidation state transition metal, a group 6, 7, 8, or 9 transition metal, chromium, molybdenum, tungsten, manganese, rhenium, iron, ruthenium, osmium, cobalt, rhodium, iridium, nickel, palladium, platinum, copper, silver, or gold.

5. The catalyst of claim 4 wherein R1 is CH.sub.3CN; R8 is CH(CH.sub.3).sub.2; R9 is CH.sub.3; R10 is CH(CH.sub.3).sub.2; and M is Ruthenium giving Formula V

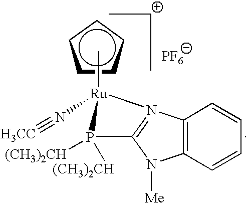

6. A method for synthesizing the catalyst of claim 4 using the steps of:
(a) utilizing a precursor containing a cyclopentadienyl ligand and a metal ion;
(b) reacting the precursor with an alternative ligand comprising a structure of 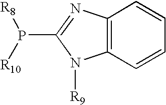
wherein the metal ion is selected from the group consisting of a transition metal, a 1+, 2+, or 3+ oxidation state transition metal, a group 6, 7, 8, or 9 transition metal, chromium, molybdenum, tungsten, manganese, rhenium, iron, ruthenium, osmium, cobalt, rhodium, iridium, nickel, palladium, platinum, copper, silver and gold.

7. A catalyst of Formula III: 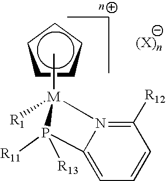
wherein R1 is selected from the group consisting of CH.sub.3CN or derivatives thereof, halide, hydride, carboxylate, sulfonate, or any substituted derivatives thereof, or any neutral or anionic ligand; R11 is selected from the group consisting of CH(CH.sub.3).sub.2, C(CH.sub.3.).sub.2, or any alkyl or aryl group, including heteroaryl; R12 is selected from the group consisting of C(CH.sub.3).sub.3, H, CH(CH.sub.3).sub.2, or any alkyl or aryl group, including heteroaryl; R13 is selected from the group consisting of CH(CH.sub.3).sub.2, C(CH.sub.3.).sub.2, or any alkyl or aryl group, including heteroaryl; and M is selected from the group consisting of a transition metal, a 1+, 2+, or 3+ oxidation state transition metal, a group 6, 7, 8, or 9 transition metal, chromium, molybdenum, tungsten, manganese, rhenium, iron, ruthenium, osmium, cobalt, rhodium, iridium, nickel, palladium, platinum, copper, silver, or gold.

8. The catalyst of claim 7 wherein R1 is CH.sub.3CN; R11 is CH(CH.sub.3).sub.2; R13 is CH(CH.sub.3).sub.2: and M is Ruthenium, giving Formula VI:

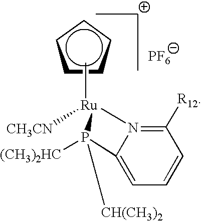

9. A method for synthesizing the catalyst of claim 7 using the steps of:
(a) utilizing a precursor containing a cyclopentadienyl ligand and a metal ion;
(b) reacting the precursor with a pyrid-2-yl phosphine ligand;
wherein the metal ion is selected from the group consisting of a transition metal, a 1+, 2+, or 3+ oxidation state transition metal, a group 6, 7, 8, or 9 transition metal, chromium, molybdenum, tungsten, manganese, rhenium, iron, ruthenium, osmium, cobalt, rhodium, iridium, nickel, palladium, platinum, copper, silver and gold.

10. A method for using catalysts selected from the group consisting of Formula, I, Formula II, Formula III, Formula IV, Formula V, Formula VI and Formula VII, wherein said method comprises contacting an hydrocarbon species with one of said catalysts under suitable reaction conditions.

11. The method of claim 10 wherein the hydrocarbon is an alkenol having the alkene and alcohol groups far apart and the catalyst moves the double bond across numerous carbon atoms.

12. The method of claim 11 wherein the catalyst moves the double bond across 8 carbon atoms.

13. The method of claim 10 wherein the hydrocarbon is an achiral alkenol and the catalyst forms a chiral alcohol therefrom.

14. The method of claim 10 wherein deuterated water is substituted in to the isomerization reaction mixture for forming deuterated hydrocarbon species.

15. A catalyst of Formula VII: 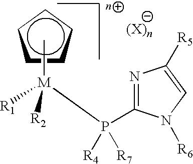
wherein R1 is selected from the group consisting of CH.sub.3CN or derivatives thereof, halide, hydride, carboxylate, sulfonate, or any substituted derivatives thereof, or any neutral or anionic ligand; R2 is selected from the group consisting of CH.sub.3CN or derivatives thereof, halide, hydride, carboxylate, sulfonate, or any substituted derivatives thereof, or any neutral or anionic ligand; R4 is selected from the group consisting of CH(CH.sub.3).sub.2, C(CH.sub.3.).sub.2, or any alkyl or aryl group, including heteroaryl; R5 is selected from the group consisting of C(CH.sub.3).sub.3, H, CH(CH.sub.3).sub.2, or any alkyl or aryl group, including heteroaryl; R6 is selected from the group consisting of CH.sub.3, H, or any alkyl or aryl group; R7 is selected from the group consisting of CH(CH.sub.3).sub.2, C(CH.sub.3.).sub.2, or any alkyl or aryl group, including heteroaryl; and M is selected from the group consisting of a transition metal, a 1+, 2+, or 3+ oxidation state transition metal, a group 6, 7, 8, or 9 transition metal, chromium, molybdenum, tungsten, manganese, rhenium, iron, ruthenium, osmium, cobalt, rhodium, iridium, nickel, palladium, platinum, copper, silver, or gold.

16. The catalyst of claim 1 wherein R1 is CH.sub.3CN; R2 is CH.sub.3CN; R4 is CH(CH.sub.3).sub.2; R5 is CH(CH.sub.3).sub.3; R6 is CH.sub.3; R7 is CH(CH.sub.3).sub.2; and M is Ruthenium.

17. A method of synthesizing a catalyst of claim 15 using the steps of:
(a) utilizing a precursor containing a cyclopentadienyl ligand and a metal ion;
(b) reacting the precursor with an imidazol-2-yl phosphine ligand;
wherein the metal ion is selected from the group consisting of a transition metal, a 1+, 2+, or 3+ oxidation state transition metal, a group 6, 7, 8, or 9 transition metal, chromium, molybdenum, tungsten, manganese, rhenium, iron, ruthenium, osmium, cobalt, rhodium, iridium, nickel, palladium, platinum, copper, silver and gold.

18. A method of synthesizing the catalyst of claim 2 using the steps of:
(a) utilizing a precursor containing a cyclopentadienyl ligand and a ruthenium(2+) ion; and
(b) reacting the precursor with an imidazol-2-yl phosphine ligand.

19. A method of synthesizing a catalyst of claim 5 using the steps of:
(a) utilizing a precursor containing a cyclopentadienyl ligand and a ruthenium(2+) ion; and
(b) reacting the precursor with an alternative ligand comprising a structure of

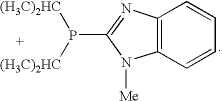

20. A method of synthesizing catalysts using the steps of:
(a) utilizing a precursor containing a cyclopentadienyl ligand and a metal ion;
(b) reacting the precursor with a ligand selected from the group consisting of an imidazol-2-yl phosphine ligand and a pyrid-2-yl phosphine ligand;
wherein the metal ion is selected from the group consisting of a transition metal, a 1+, 2+, or 3+ oxidation state transition metal, a group 6, 7, 8, or 9 transition metal, chromium, molybdenum, tungsten, manganese, rhenium, iron, ruthenium, osmium, cobalt, rhodium, iridium, nickel, palladium, platinum, copper, silver and gold.
